(19) United States
(12) Patent Application Publication
(10) Pub. No.: US 2016/0106699 A1
(43) Pub. Date: Apr. 21, 2016

(54) Oral pharmaceutical composition containing combination of PPARa and a HMG-CoA reductase inhibitor

(71) Applicants: Arthur Deboeck, (US); Francis Vanderbist, Beersel (BE); Philippe Baudier, Uccle (BE)

(72) Inventors: Arthur Deboeck, (US); Francis Vanderbist, Beersel (BE); Philippe Baudier, Uccle (BE); Antonio Sereno, Melsbroek (BE)

(73) Assignee: Galephar Pharmaceutical Research, Inc.

(21) Appl. No.: 12/385,758

(22) Filed: Apr. 17, 2009

Related U.S. Application Data
(63) Continuation of application No. 10/486,219, filed on Sep. 8, 2004, now abandoned.

(30) Foreign Application Priority Data
Aug. 7, 2001 (BE) PCT/BE01/00133
Sep. 7, 2001 (BE) PCT/BE01/00147

(51) Int. Cl. A61K 31/235; A61K 31/22; A61K 31/365; A61K 9/48; A61K 9/00

(52) U.S. Cl. CPC A61K 31/235; A61K 9/4866; A61K 9/4858; A61K 9/0053; A61K 31/365; A61K 31/22

(57) ABSTRACT

Oral pharmaceutical composition containing, in the same pharmaceutical form, effective amounts of a HMG-CoA reductase inhibitor derivative and of PPARα, especially fenofibrate. Also described is the use of some inactive ingredients which allow to improve the dissolution and/or bioavailability of the drugs from the said composition.

### BACKGROUND OF THE INVENTION

Hypercholesterolaemia plays a crucial role in the development of atherosclerosis diseases in general and coronary heart disease in particular. The risk of progression of the atherosclerosis process to coronary heart diseases increases progressively with increasing levels of total serum cholesterol or low density lipoproteins (LDL) cholesterol at both the individual and the population level.

The HMG-CoA reductase inhibitors are reversible inhibitors of the microsomal enzyme. HMG-CoA reductase, which converts HMG-CoA to mevalonate. This is an early rate-limiting step in cholesterol biosynthesis. Inhibition of HMG-CoA reductase by HMG-CoA reductase inhibitors decreases intracellular cholesterol biosynthesis, which then leads to transcriptionnally upregulated production of microsomal HMG-CoA reductase at cell surface LDL receptors.

Subsequently, additional cholesterol is provided to the cell by de novo synthesis and by receptor-mediated uptake of LDL-cholesterol from the blood. This resets intracellular cholesterol homeostasis in extrahepatic tissues, but has little effect on the overall cholesterol balance (Clin. Pharmacokinet. 1997, May, 32(5), 403-425).

The main HMG-CoA reductase inhibitors currently used in therapeutics are: pravastatin, simvastatin, lovastatin, fluvastatin, atorvastatin and cerivastatin. simvastatin, lovastatin and pravastatin are derived from fungi (14,15).

simvastatin reductase inhibitor is a clinically modified 2,2-dimethyl-butyrate analogue of lovastatin.

Fibrates are old hypolipidemic drugs with pleitropic effects on lipid metabolism. Their intimate molecular mechanisms of action have been mysterious for a long time. Recently, it has been shown that the pharmacological effect of fibrates depends on their binding to “Peroxisome Proliferator Activated Receptor alpha” (PPAR alpha). The binding of fibrates to PPAR induces the activation of the inhibition of multiple genes involved in lipid metabolism through the binding of the activated PPAR alpha to is “Peroxisome Proliferator Response Element” (PPRE) located in the gene promoters. Furthermore, it was recently demonstrated that fibrates are potent antiinflammatory molecules through an indirect modulation of the nuclear-factor-Kappa B activity.

Fenofibrate or P-(4-chlorobenzoyl)-phenoxy isobutyrate isopropyl ester is useful for the treatment of adult patients with very high elevations of serum triglyceride levels and/or cholesterol levels. The usual daily dosage is 100 to 300 mg which is administered in two or three doses. Fenofibrate is absorbed as fenofibric acid which is responsible for the pharmacological activity. Fenofibric acid resulting from the hydrolysis of fenofibrate is extensively bound to plasma albumin. The plasma half-life is about 20 hours. Fenofibric acid is excreted predominantly in the urine, mainly as the glucuronide conjugate, but also as a reduced form of fenofibric acid and its glucuronides.

It has been demonstrated that the combination of a HMG-CoA reductase inhibitor and fenofibrate (administered in two separate dosage forms) was better tolerated and as efficient as a higher dose of the HMG-CoA reductase inhibitor derivative. The main disadvantage of this double administration is that it complicates the posology for the patients and hence it increases the risk of mistakes or omissions in the intake of drugs. The patient's compliance is then decreased.

Consequently, there is still a need for patients suffering from hypercholesterolemia and/or lipidemia to dispose of a pharmaceutical dosage form containing effective amounts of at least one HMG-CoA to reductase inhibitor derivative and of fenofibrate and allowing to obtain a good bioavailability of both drugs.

Some patents describing association of hypolipidemiant agents are already described. For instance, U.S. Pat. No. 6,180,660 describes methods for preventing or reducing the risk of a first occurrence of a cardiovascular event using an HMG-CoA reductase inhibitor alone or in combination with another lipid altering agent. Subjects to be treated are those having an average serum total cholesterol level, an average to mildly elevated serum low-density lipoprotein cholesterol level, and a below average serum high-density lipoprotein cholesterol level, with no history of clinically evident coronary disease.

The U.S. Pat. No. 6,264,938 relates to methods for treating hypercholesterolemia and atherosclerosis, and reducing serum cholesterol in a mammal. The methods of the invention comprise administering to a mammal a first amount of a bile acid sequestrant compound which is an unsubstituted polydiallylamine polymer and a second amount of an HMG CoA reductase inhibitor compound. The first and second amounts together comprise a therapeutically effective amount. The invention further relates to pharmaceutical compositions useful for the treatment of hypercholesterolemia and atherosclerosis, and for reducing cholesterol.

The WO 01/37831 describes a pharmaceutical combination comprising separate dosage form in a common blister card of an inhibitor of the HMG-CoA reductase and a fibric acid derivative useful in the treatment at different ways of dyslipidemia of diabetics and non-diabetics.

U.S. Pat. No. 5,545,628 describes an advantageous oral pharmaceutical composition containing fenofibrate while the patent PCT/BE 01/00098 describes an advantageous semi-solid oral pharmaceutical composition containing an HMG-CoA reductase.

Not described is an oral semi-solid pharmaceutical composition containing, in the same pharmaceutical form, a combination of an effective amount of a HMG-CoA reductase inhibitor derivative together with an effective amount of a PPARα agent, especially fenofibrate.

### SUMMARY OF THE INVENTION

The present invention relates to an oral pharmaceutical composition, containing a combination of effective amounts of at least one HMG-CoA reductase inhibitor derivative and of PPARα agent, especially fenofibrate, in the same dosage form, allowing to obtain a high bioavailability of all drugs. The invention also relates to a process for manufacturing the same.

### DETAILED DESCRIPTION OF THE INVENTION

It is an object of the present invention to disclose a pharmaceutical dosage form containing PPARα agent, especially fenofibrate, and at least a HMG-CoA reductase inhibitor of the statin family.

It is another object of the present invention to disclose a pharmaceutical dosage form containing PPARα agent, especially fenofibrate, and at least a HMG-CoA reductase inhibitor contained in a capsule or a tablet.

Another object of the present invention is to disclose a pharmaceutical dosage form containing PPARα agent, especially fenofibrate, and at least a HMG-CoA reductase inhibitor with increased bioavailability for both the PPARα agent, especially fenofibrate, and all the HMG-CoA reductase inhibitor.

Also an object of the present invention is to disclose a pharmaceutical dosage form containing PPARα agent, especially fenofibrate, and at least a HMG-CoA reductase inhibitor from which at least the PPARα agent, especially fenofibrate, has an increased bioavailability.

Also an object of the present invention is to disclose a pharmaceutical dosage form containing PPARα agent, especially fenofibrate, and at least a HMG-CoA reductase inhibitor from which at least one of the HMG-CoA reductase inhibitors have an increased bioavailability.

The formulation contains advantageously at least one hydrophilic agent (HLB>10) and/or one or more stabilizing agent(s) e.g. one or more antioxidant or preservative agent or a combination of both preservative and antioxidant agents.

According to the invention, a pharmaceutical composition useful for administration to a mammal comprises in a same dosage form an effective amount of at least one peroxisome proliferator activated agent (PPARα), an effective amount of at least one HMG-CoA reductase inhibitor derivative of the statin family, and at least one polyglycolized glyceride or another derivative of glyceride.

Preferably, the composition comprises at least one peroxisome proliferator activated agent (PPARα) under the form of semi-solid composition (containing at least one polyglycolized glyceride or another derivative of glyceride) and at least one HMG-CoA reductase inhibitor derivative of the statin family under the form of a coated tablet, both the semi-solid form and the tablet being filled in one single pharmaceutically acceptable capsule

Preferably, the composition further comprises at least one hydrophilic disintegrating agent. Examples of disintegrating agents are sodium starch glycolate, sodium croscarmellose, crospovidone, starch, colloidal silicone dioxide or another pharmaceutically accepted disintegrating agent and combinations thereof, sodium starch glycolate being preferred.

In the composition of the invention, the PPARα agent is advantageously a compound of the fibrate family, preferably a compound selected from the group consisting of fenofibrate, ciprofibrate, clofibrate, gemfibrozil, bezafibrate and combinations thereof. Especially, the PPARα agent is fenofibrate.

The pharmaceutical composition of the invention is preferably in a form suitable for the oral administration of the active agents.

The polyglycolised glyceride has advantageously an HLB balance above 10, preferably above 11, most preferably above 12.

According to an embodiment, the melting point of the said composition is below 90° C., preferably below 80° C., most preferably below 70° C.

The pharmaceutical composition of the invention contains advantageously one or more antioxidant and/or preservative agent(s), such as a vitamin E derivative and/or a methoxyphenol derivative and/or a combination thereof.

The pharmaceutical composition of invention contains advantageously a wetting agent.

The composition of the invention further contains advantageously a polyethylene glycol or a mix of polyethylene glycol with different molecular mass; and/or a suspension stabilizer, such as a cellulose derivative, hydropropylcellulose.

The composition of the invention is adapted for the administration of specific amount of active agent per dose. Advantageously, the amount of PPARα, preferably fenofibrate, per dose is between 30 and 400 mg, while the amount of statin per dose is between 5 and 100mg, the amount of statin per dose being preferably lower than the amount of PPARα per dose, most preferably comprised between 0.01 and 0.5 times the amount of PPARα per dose.

The composition of the invention is for example filled in hard gelatine capsules, hypromellose capsules or in other pharmaceutically acceptable capsules.

According to a preferred detail of the composition, the composition is with the proviso that the PPARα, preferably fenofibrate, is not co-micronized, and/or, preferably and, with the proviso that the statin is not co-micronized.

According to preferred embodiments, the weight ratio PPAR agent+statin/hydrophilic disintegrating agent is comprised between 100 and 0.1, advantageously between 50 and 2, preferably between 40 and 4, more preferably between 6 and 25, such as 8, 10, 12, 14, etc.

According to a further detail of a preferred embodiment, the weight ratio PPAR+statin agent/polyglycolized glyceride(s) is comprised between 10 and 0.1, advantageously between 5 and 0.2, preferably lower than 1, more preferably between 0.8 and 0.3, such as about 0.8, 0.7, 0.6, 0.5, 0.4.

For example, at least one HMG-CoA reductase inhibitor derivative of the statin family is advantageously selected from the group consisting of pravastatin, simvastatin, lovastatin, fluvastatin, atorvastatin and cerivastatin. simvastatin, lovastatin, pravastatin and mixtures thereof.

The pharmaceutical composition of the invention advantageously further contains a polyethyleneglycol derivative (PEG). The amount of PEG is advantageously comprised between 0.2 and 5 times the amount of stain present in the composition, preferably between 0.5 and 2 times the amount of statin present in the composition.

According to a specific embodiment, the composition contains one or more antioxidant and/or preservative agent(s), one polyethylene derivative, and one hydrophilic wetting agent.

The semi-solid composition may be a suspension, an emulsion or a micro-emulsion. The HMG-CoA reductase inhibitor agent and the fibric acid derivative may be partially or totally dissolved in the semi-solid matrix formed by the excipients.

The advantages of the semi-solid formulations are multiple for HMG-CoA reductase inhibitors: protection of the active ingredient from air and humidity, possibility of increasing the dissolution rate of the molecule and hence of bioavailability, diminution of the risk of contamination of the operator, diminution of the risk of cross contamination, no possibility of demixing under the effect of vibrational mixing during manufacturing process, facility of the production process. The choice of the nature of the formulation of course influenced the stability of the pharmaceutical form and the bioavailability of the drug contained in it. Generally, a maximum bioavailability is achieved by preparing and keeping the drug in the amorphous/solubilized state in a solid dispersion or in a lipid-based formulation. For these systems, the barrier we are avoiding is the compound <<washing-out>> of solution to a large extent into a insoluble crystalline form during the dissolution/release step in vivo.

These systems may consist of suspension, emulsion, microemulsion, self-emulsifying drug delivery systems (SEDDS) or self-emulsifying microemulsion drug delivery system (SMEDDS).

Microemulsions have the added advantage over suspensions such as emulsions and dispersions since thermodynamically they are more stable, that they can be manufactured with little energy input and have generally a longer shelf-life.

The formation of oil-in-water (O/W) and water-in-oil (W/O) microemulsions usually involves a combination of 3-5 basic compounds i.e. oil, surfactant, cosurfactant, water and electrolytes. The challenge is to select for a particular application oil(s) and surfactant(s) that are acceptable from a toxicological perspective and that allow to obtain a high bioavailability of the drug.

The assessment of the quality of semi-solid lipid based formulations is quite difficult since the in vitro dissolution test is of little help. Indeed, the in vitro/in vivo correlation between dissolution and bioavailability is very poor for this kind of formulation. Other analytical tools are available to the formulator to try to predict the in vivo bioavailability of isotretinoin from various formulations like the CACO-2 cells model, the assessment of the percentage of drug dissolved in the formulation, differential scanning calorimetry, microscopy, . . . .

Nevertheless, none of them present a guarantee of in vitro/in vivo correlation and ultimately only pharmacokinetic studies on human subjects are reliable to assess the bioavailabiltiy of the drug.

Advantageously, the melting point of the final composition will be below 80° C., preferably below 60° C.

Advantageously, an emulsifier may be added (e.g distilled monoglycerides, Myverol®, Gillco, US) to the formulation in order to increase the solubilization of the HMG-CoA reductase inhibitor.

Advantageously, the oral pharmaceutical composition may contain a solubilizing agent. This solubilizing agent is advantageously water and HCl soluble. An example of this kind. of solubilizing is diethylene glycol monoethyl ether (Transcutol®, Gattefossé).

Also advantageous for the stability and the bioavailability of the composition is the addition of an antioxidant agent such as either a Tocopherol derivative like Tocopherol (Vitamine E), Tocopherol acetate, Vitamine E TPGS or a methylphenol derivative like butylhydroxyanisol (BHA) or butylhydroxytoluene (BHT).

The addition of a polymer able to control the recristallisation of the active ingredient may also be useful when the active ingredient is not completely dissolved in the semi-solid matrix.

The role of the polymer is (i) to stabilize the semi-solid formulation by increasing the viscosity of the composition and (ii) to avoid the growth of particles of active ingredient that are not solubilized (or formed during the cooling of the composition) by forming a matrix in the semi-solid composition.

Examples of such agents are cellulose derivatives such as hydroxypropylcellulose, hypromellose and methylcellulose.

A wetting agent may also be added advantageously to the said composition when a very fast release in needed. Example of such agents are Na croscarmellose, Na carboxymethylcellulose or reticulated povidone. The effect of the wetting agent is strongly dependent on the nature of the active ingredient and on the nature of the semi-solid matrix.

Process for manufacturing the said pharmaceutical composition.

One of the advantages of the invention relates to the easiness of the manufacturing process of the medication and the rapidity and easiness of the pharmaceutical composition.

Briefly, the inactive ingredients are used as molten together. In an adequate tank the active ingredient is added to the molten mass and once the solution mass is homogenous, the molten is filled into pharmaceutically acceptable capsules e.g. hard gelatin capsules or hypromellose capsules. The capsules are then cooled and thereafter adequately packaged.

### EXAMPLES OF FORMULATIONS

### Example 1

| | mg/capsule | | |
|---|---|---|---|
| Ingredient | F1 | F2 | F3 |
| Simvastatin | 20 | 15 | 12 |
| Fenofibrate | 200 | 160 | 160 |
| Gelucire 44/14 ® | 350 | 300 | 400 |
| Vit E TPGS | 20 | 30 | 30 |
| Polyethyleneglycol 6000 | 30 | 20 | 30 |
| Butylhydroxyanisole | 0.08 | 0.04 | 0.08 |
| Propyl gallate | — | 0.04 | — |

### Example 2

| | mg/capsule | | |
|---|---|---|---|
| Ingredient | F4 | F5 | F6 |
| lovastatin | 15 | 15 | 12 |
| Fenofibrate | 150 | 150 | 160 |
| Gelucire 44/14 ® | — | 350 | — |
| Gelucire 50/13 | 350 | — | 400 |
| PEG 6000 | — | — | 20 |
| PEG 2000 | — | 20 | 10 |
| Butylhydroxyanisole | 0.04 | — | 0.04 |
| Butylhydroxytoluene | 0.04 | 0.08 | 0.04 |

### Example 3

| Ingredient | mg/capsule |
|---|---|
| semi-solid formulation | |
| Fenofibrate | 160 |
| hydropropylcellullose | 95 |
| Gelucire 44/14 ® | 350 |
| PEG 6000 | 60 |
| uncoated tablet | |
| Pravastatin | 12.5 |
| Mannitol | 70 |
| Lactose | 15 |
| Microcrystalline cellulose | 11.5 |
| Sodium starch glycolate | 9 |
| butylhydroxyanisole | 0.01 |
| Magnesium Stearate | 2 |
| Coating of the tablet | |
| Absolute ethanol | 36.46 |
| Povidone | 7.29 |
| Talc | 3.64 |
| Triacetin | 0.607 |
| pro capsula una | |

The example 3 hereinabove describes a composition corresponding to the present invention wherein the fenofibrate is formulated as a semi-solid formulation and the pravastatin as a coated tablet formulation, the fenofibrate and pravastatin formulations being filled as a single composition in one single hard gelatin capsule.

### Example 4

| | mg/capsule | | |
|---|---|---|---|
| Ingredient | F7 | F8 | F9 |
| pravastatin | 30 | 40 | 25 |
| Fenofibrate | 160 | 200 | 160 |
| Gelucire 44/14 ® | 350 | 300 | 300 |
| PEG 6000 | 20 | 20 | 30 |
| Vit E TPGS | 30 | 20 | 30 |
| Sodium starch glycolate | 20 | 10 | 20 |
| Lactose | — | 10 | — |
| Butylhydroxyanisole | 0.08 | 0.08 | 0.08 |
| Hydroxypropylcellulose | — | — | 100 |

In the present specification, the term “improved bioavailability” relates to the human bioavailability of the drug(s) in humans. The bioavailability of a drug is defined as the rate and extent to which the active substance or active moiety is absorbed from a pharmaceutical form and becomes active at the site of action. The bioavailability is essentially quantified by the area under the plasma concentration curve (AUC) and the maximal plasma concentration (Cₘₐₓ). Consequently, an improved form of the invention to presents a higher bioavailability (AUC and/or Cₘₐₓ), preferably a significantly higher bioavailability than the reference, namely the actually commercialized fenofibrate form or/and the actually commercialized HMG-CoA reductase form, the drug being taken via the oral route at the same dose. The preferred form of the invention presents a higher bioavailability is (AUC and/or Cₘₐₓ), preferably a significantly higher bioavailability than the references which are respectively the actually commercialized form of fenofibrate and the actually commercialized form of HMG-CoA reductase inhibitor, when the products are taken via the oral route at the same dose. The improved bioavailability is for example improved of at least 10%, advantageously of at least 15%, preferably of at least 20% with respect to the bioavailability of the reference.

The compositions of the various formulations F1 to F9 of examples 1 to 3 have been repeated, except that the fenofibrate has been replaced by ciprofibrate, clofibrate, gemfibrozil, bezafibrate, a combination of bezafibrate (50%) and fenofibrate (50%).

1-33. (canceled)

34. A pharmaceutical composition comprising in a same dosage form an amount of at least one peroxisome proliferator activated compound (PPARα), an amount of at least one HMG-CoA reductase inhibitor compound which is a statin family, and at least one polyglycolized glyceride.

35. The pharmaceutical composition of claim 34, wherein the PPARα compound is contained in a semi-solid vehicle comprising at least least one polyglycolized glyceride, and the statin compound is formulated as a tablet, the formulations of both being filled into one single pharmaceutically acceptable capsule.

36. The pharmaceutical composition of claim 34, wherein the PPARα compound is contained in a semi-solid vehicle comprising at least least one polyglycolized glyceride, and the statin compound is formulated as a coated tablet, the formulations of both being filled into one single pharmaceutically acceptable capsule.

37. The pharmaceutical composition of claim 34, which comprises at least one hydrophilic disintegrating compound.

38. The pharmaceutical composition of claim 34, wherein the PPARα compound is a fibrate compound.

39. The pharmaceutical composition of claim 34, wherein the PPARα compound is fenofibrate.

40. The pharmaceutical composition of claim 34, which is for oral administration.

41. The pharmaceutical composition of claim 40, wherein the polyglycolised glyceride has an HLB balance above 10.

42. The pharmaceutical composition of claim 34, wherein the melting point of the composition is below 90° C.

43. The pharmaceutical composition of claim 34, containing at least one further compound selected from the group consisting of antioxidants and preservatives.

44. The pharmaceutical composition of claim 34, further comprising a vitamin E compound.

45. The pharmaceutical composition of claim 34, containing a methoxyphenol compound.

46. The pharmaceutical composition of claim 43, where a combination of a vitamin E compound and a methoxyphenol compound is used as antioxidant and preservative agent, respectively.

47. The pharmaceutical composition of claim 34, wherein the composition contains a wetting compound.

48. The pharmaceutical composition of claim 37, wherein the disintegrating compound is sodium starch glycolate.

49. The pharmaceutical composition of claim 37, wherein the at least one hydrophilic disintegrating compound is selected from the group consisting of sodium croscarrnellose, crospovidone, starch, and colloidal silicone dioxide.

50. The pharmaceutical composition of claim 34, further comprising polyethylene glycol.

51. The pharmaceutical composition of claim 34, further comprising a suspension stabilizer.

52. The pharmaceutical composition of claim 51, wherein the suspension stabilizer is a cellulose compound.

53. The pharmaceutical composition of claim 52, wherein the cellulose compound is hydropropylcellulose.

54. The pharmaceutical composition of claim 34, wherein the amount of PPARα compound per dose is between 30 and 400 mg, while the amount of statin compound per dose is between 5 and 100 mg.

55. The pharmaceutical composition of claim 35, wherein the pharmaceutically-acceptable capsule is selected from the group consisting of hard gelatin capsules, and hypromellose capsules.

56. The pharmaceutical composition of claim 36, wherein the pharmaceutically-acceptable capsule is selected from the group consisting of hard gelatin pharmaceutical capsules, and hypromellose capsule.

57. The pharmaceutical composition of claim 34, with the proviso that the PPARα compound is not co-micronized.

58. The pharmaceutical composition of claim 34, comprising at least one disintegrating compound, in which the weight ratio of PPARα, compound +statin/hydrophilic disintegrating agent is between 100 and 0.1.

59. The pharmaceutical composition of claim 35, in which the weight ratio of PPARα compound +statin agent/polyglycolized glyceride(s) is between 10 and 0.1.

60. The pharmaceutical composition of claim 34, wherein the at least one HMG-CoA reductase inhibitor compound is selected from the group consisting of pravastatin, simvastatin, lovastatin, fluvastatin, atorvastatin and cerivastatin. simvastatin, lovastatin, pravastatin and mixtures thereof.

61. The pharmaceutical composition of claim 34, which further comprises a polyethyleneglycol compound.

62. The pharmaceutical composition of claim 34, which further comprises one or more antioxidant or preservative compounds, or both a polyethylene compound, and a hydrophilic wetting compound.

63. A method of treating hyperlipidemia or hypercholesterolemia or both in human in need thereof, which comprises administering orally substantially simultaneously, an effective amount of at least one peroxisome proliferator activated compound (PPARα), an effective amount of at least one HMG-CoA reductase inhibitor compound of the statin family, and at least one glyceride compound.

64. The method of claim 63, wherein the at least one glyceride compound is polyglycolized glyceride.

65. The method of claim 63, which further comprises administering at least one hydrophilic disintegrating compound.

66. The method of claim 63, wherein the PPARα compound is a compound of the fibrate family.

67. The method of claim 66, wherein the PPARα compound is selected from the group consisting of fenofibrate, cipofibrate, clofibrate, gemfibrate, bezafibrate and combination thereof.

68. The method of claim 63, wherein the PPARα compound is fenofibrate.

69. The method of claim 63, wherein at least one HMG-CoA reductase inhibitor compound of the statin family is selected from the group consisting of pravastatin, simvastatin, lovastatin, fluvastatin, atorvastatin and cerivastatin. simvastatin, lovastatin, pravastatin and mixtures thereof.

70. The pharmaceutical composition of claim 34, wherein the PPARα compound is a compound selected from the group consisting of fenofibrate, ciprofibrate, clofibrate, gemfibrozil, bezafibrate and mixtures thereof.

71. The pharmaceutical composition of claim 34, wherein the polyglycolized glyceride has an HLB balance above 11.

72. The pharmaceutical composition of claim 71, wherein the polyglycolized glyceride has an HLB balance above 12.

73. The pharmaceutical composition of claim 34, wherein the melting point of the composition is below 80° C.

74. The pharmaceutical composition of claim 34, wherein the melting point of the said composition is below 70° C.

75. The pharmaceutical composition of claim 34, further containing a mixture of polyethylene glycols having different molecular masses.

76. The pharmaceutical composition of claim 34, wherein the amount of PPARα compound per dose is between 30 and 400 mg, while the amount of statin per dose is between 5 and 100 mg, the amount of statin per dose being lower than the amount of PPARα compound per dose.

77. The pharmaceutical composition of claim 34, wherein the amount of PPARα compound per dose is between 30 and 400 mg, while the amount of statin per dose is between 5 and 100 mg, the amount of statin per dose being between 0.1 and 0.5 times the amount of PPARα compound per dose.

78. The pharmaceutical composition of claim 34, wherein the amount of fenofibrate per dose is between 30 and 400 mg, while the amount of statin per dose is between 5 and 100 mg.

79. The pharmaceutical composition of claim 34, wherein the amount of fenofibrate per dose is between 30 and 400 mg, while the amount of statin per dose is between 5 and 100 mg, the amount of statin per dose being preferably lower than the amount of fenofibrate per dose.

80. The pharmaceutical composition of claim 34, wherein the amount of fenofibrate per dose is between 30 and 400 mg, while the amount of statin per dose is between 5 and 100 mg, the amount of statin per dose being between 0.1 and 0.5 times the amount of fenofibrate per dose.

81. The pharmaceutical composition of claim 34, with the proviso that the PPARα compound is not co-micronized, and with the proviso that the statin is not co-micronized.

82. The pharmaceutical composition of claim 34, with the proviso that fenofibrate is not co-micronized, and with the proviso that the statin is not co-micronized.

83. The pharmaceutical composition of claim 34, comprising at least one disintegrating compound, in which the weight ratio PPARα compound+statin/hydrophilic disintegrating compound is between 50 and 2.

84. The pharmaceutical composition of claim 34, comprising at least one disintegrating compound, in which the weight ratio PPARα compound+statin/hydrophilic disintegrating compound is between 40 and 4.

85. The pharmaceutical composition of claim 34, comprising at least one disintegrating compound, in which the weight ratio PPARα compound+statinlhydrophilic disintegrating compound is between 6 and 25.

86. The pharmaceutical composition of claim 34, comprising at least one disintegrating compound, in which the weight ratio PPARα compound statinlhydrophilic disintegrating compound is between 50 and 2.

87. The pharmaceutical composition of claim 34, comprising at least one disintegrating compound, in which the weight ratio PPARα compound statin/hydrophilic disintegrating compound is between 40 and 4.

88. The pharmaceutical composition of claim 34, comprising at least one disintegrating compound, in which the weight ratio PPARα compound+statin/hydrophilic disintegrating compound is between 6 and 25.

89. The pharmaceutical composition of claim 34, in which the weight ratio PPARα compound+statin compound/polyglycolized glyceride(s) is between 5 and 0.2.

90. The pharmaceutical composition of claim 34, in which the weight ratio PPARα compound+statin compoundlpolyglycolized glyceride(s) is between 0.8 and 0.3.

91. The method of claim 63, wherein the PPARα compound is a compound selected from the group consisting of fenofibrate, ciprofibrate, clofibrate, gemfibrozil, bezafibrate and mixtures thereof.
